(19) United States
(12) Patent Application Publication
(10) Pub. No.: US 2021/0210197 A1
(43) Pub. Date: Jul. 8, 2021

(54) SYSTEMS AND METHODS FOR IMPROVING PATIENT SATISFACTION

(71) Applicant: Norah Health LLC, Olivette, MO (US)

(72) Inventors: Dmitry Beyder, St. Louis, MO (US); Daniel Hoffman, St. Louis, MO (US); Philip Thompson, Rockwall, TX (US)

(21) Appl. No.: 17/038,101

(22) Filed: Sep. 30, 2020

Related U.S. Application Data
(60) Provisional application No. 62/908,969, filed on Oct. 1, 2019.

(51) Int. Cl. G16H 40/20; G16H 50/20; G16H 10/60

(52) U.S. Cl. CPC G16H 40/20; G16H 10/60; G16H 50/20

(57) ABSTRACT

Systems and methods designed to capture and utilize the context of a patient's visit along with a general categorization of their personality for the purpose of identifying actions to a healthcare provider for use during the specific patient visit. The actions are intended to be used by the healthcare provider to improve the patient's overall satisfaction with their care and specifically their reported satisfaction on various patient satisfaction feedback surveys. The systems and methods also can serve to improve future patient visits through better understanding, better engagement, improved trust, and better overall experience with healthcare providers.

### CROSS-REFERENCE TO RELATED APPLICATIONS

This application claims the benefit of U.S. Provisional Patent Application Ser. No. 62/908,969, filed Oct. 1, 2019, the entire disclosure of which is herein incorporated by reference.

### BACKGROUND OF THE INVENTION

### 1. Field of the Invention

This disclosure relates to systems and methods for improving patient communication and understanding particularly in a medical setting and particularly through capturing the context of a patient visit combined with information on the patient's personality.

### 2. Description of the Related Art

There can be little doubt that modern medicine is complicated. Even relatively simple medical complaints can have complex treatments. Further, even small medical complaints can be very difficult to treat as a large number of factors can contribute to what results in the complaint. One of the ways which is believed to improve outcomes is to make sure that patients are directly involved in their own care and understand what is happening in their individual case. While it can be difficult for patients to appreciate the nuances of care specifics, patients are typically much more aware of their own feelings with regards to the care than any care provider is. Understanding and correctly recognizing these feelings can be highly important to successful care arrangements. Gone are the days where care was simply foisted on a patient “for their own good.” Instead, patients are actively engaged during the care process to make sure they understand why and how suggestions are being made for care.

This process is often referred to as “patient engagement.” Patient engagement is designed to encourage patients in their own care, make sure they are engaged during office visits in a comfortable and understandable manner, and to insure that they are interacted with in a medical setting in a way that appeals to their own sensibilities and values. Patient engagement systems typically pair a patient's knowledge and willingness to participate in their own care, and to deal with an underlying medical situation in a way that suits them, with communications designed to promote positive behaviors and facilitate interactions with healthcare professionals. In effect, patients now are commonly given more control over their care, or are at least given a better understanding of what care needs to be.

Patient engagement is used by many healthcare organizations to improve patient outcomes through improving the comfort and relationship of the patient during the lifecycle of care. One element of patient engagement is to recognize that different patients may have different desires when it comes to their care. This is both in how the care may be provided as well as what a patient expects and desires to be informed about with regards to a specific procedure or medical action. These specific elements may be referred to as “patient preferences” and essentially contemplate the individual's evaluation of the specifics of care and health outcomes. Patient preferences can cover a wide range of different elements, but typically relate simply to how a patient expects, and wants, to be treated. For example, some patients are interested in getting large amounts of raw data on a medical condition and make a decision on care with little input from a professional while others may wish to simply be told the best course of action from a provider's professional judgement and accept that.

As much as one would expect that treating a patient how they wish to be treated would result in improved patient satisfaction with the result, research on the use of patient preferences in the prescribing of care has actually shown mixed results in driving better outcomes [Street, et al. Patient Healthcare and Outcomes: An Ecological perspective. 2015]. The reasons for this likely lie within the prevue of decision making and the deeply personal outcome associated with medical care. In the first instance, healthcare professionals operate under a number of potentially conflicting mandates and in a realm of uncertainty. Conflicting mandates include the need to provide care in a sustainable (e.g. profitable) fashion, to provide care which is effective and within regulatory guidelines, and to provide care which complies with what a patient actually says they want as well as what they may actually want without realizing it. Sometimes these mandates can directly conflict. For example, if a patient wants to refuse certain forms of treatment (which they really should have) for financial or religious reasons, a patient could have a very negative health outcome (which could be prevented) if their desires are followed.

Recognizing and enabling a patient preference for care may actually conflict with normal standard care practices or other obligations a healthcare professional has to deal with. This conflict, if patient preferences are followed, may actually result in higher costs to the patient and the healthcare provider without any change to the actual outcomes [Daily, Modern Healthcare, 2013] or, as contemplated above, to decreased health results. These outcomes can then result in financial or reputational damage to the healthcare provider as reduced effectiveness of providing care, recidivism around underlying conditions when care is provided, and increased cost. These can all result in patients looking for care elsewhere or even in reimbursement reductions from insurance providers for this or future care.

Further, providing care based on a patient's stated preference may also result in a more negative health result for that patient, which could actually lead to dissatisfaction with the care provided even though the preference was followed. This can result, for example, because a patient made a decision on care but later felt that the decision was made without being provided information that was important to them but not to the healthcare professional. Because of these and many similar issues, there is, therefore, an incentive provided by insurers (both public and private), reputational reporting systems, and other related institutions to potentially ignore patient preferences and follow established care norms in a particular case to avoid a more negative or pervasive problem both for this patient and for future relationships with other patients.

In the alternative, however, providing care which a patient is not interested in or is not their preferred choice can result in them being very dissatisfied with the care provider even if the outcome is highly successful. Further, small complications in such a scenario may gain increased attention and influence over a patient's reaction to the care. As much as these types of issues relate to “feelings” about care and may be discounted by some practitioners, they should not be. Understanding and involvement in care can not only alter the patient's perception of the desirability of the cam, but its actual effectiveness. A good example could be a patient that is told to take a new drug with substantial side effects. If the patient believes that taking the drug and dealing with side effects is more desirable than not taking it, they are far more likely to maintain an accurate drug regimen and not be upset with the occurrence of the known side effects. However, if the same patient has not reached their own conclusion that taking the drug is worth it, a side effect (especially an unexpected one) may derail their regimen.

Modern medicine has recognized that dissatisfaction with care can result in decreased adherence to care and the Centers for Medicare and Medicaid Services (cms.org) has launched a national standardized survey that is administered to random patients for the sole purpose of measuring patient satisfaction. The resulting findings of patient satisfaction are also made available to the public. These publicized results have the potential to create brand and perception risk to healthcare providers (compared to other like providers) if they don't cater to patient's specific care desires as well as their care requirements. Also, these results are currently being used in the Centers for Medicare and Medicaid Services value-based payment model to influence a healthcare organization's reimbursement. Thus, healthcare providers are now stuck with having to both work within a patient's individual desire for care (and being judged for their ability to do so), while simultaneously providing the best care, which may only come at the expense of discounting a patient's preference for care and following established norms (and being judged for their willingness to do so). Further, a breakdown in either portion (which is essentially guaranteed if the patient's preference is at odds with established care) can result in potential loss of income and reputation for the healthcare provider.

These seemingly inconsistent needs are not insurmountable but have created tensions and problems for those in the healthcare fields. Specifically, healthcare organizations need good information on how to provide care to a patient in a fashion where the patient, effectively, will be able to make a decision which is at least as informed (or at least as accurate) as that made by a health professional, and often more so as they have a better understanding of themselves. To try and gauge how effective they are at supplying care to patients, healthcare organizations typically employ outside services to measure patient satisfaction and are provided periodic reports of these findings with the goal of improving patient interaction both with specific patients and with patients generally. In analyzing the use of patient satisfaction, however, patient preference information is not typically based on an understanding of the patient's specific personality or the context of the visit. Instead, patient satisfaction typically requests the patient tell the healthcare provider a more general satisfaction level with whatever care was ultimately provided or if they felt that they got an understanding of the appropriate care.

While this can be useful to diagnose a problem with patient understanding, it does very little to assist in correcting it. This is particularly true because different patients given the exact same information may have different reactions or perceptions. Given the increasing importance of patient satisfaction on a healthcare organization's perception as a quality facility, and the ongoing need to better understand the impact of patient satisfaction on positive healthcare outcomes, a system is needed that can capture a patient's expected natural preferences for the conduct of a specific visit along with the ability to determine if the visit fulfilled its purpose to the patient, not only from a healthcare outcome but also from a patient satisfaction level. Effectively, the system needs to capture satisfaction for a specific patient visit and know why that visit was either satisfactory or unsatisfactory to that patient.

Previously, systems and methods sought to capture only resultant satisfaction which serves well as a performance indicator, but often provides little insight into preferred behavior other than at a macro level. To deal with this, systems were developed that evaluated a patient personality style and attempted to use it to provide insights into healthcare engagement focused on identifying a category of personality to consider when considering a future patient visit. These systems at least attempted to be forward looking to improve satisfaction in future interactions with the patient. However, because of the nature of personality dynamics and the impact of the presence of a plurality of factors that could affect the patient during the entirety of their visit, the context of the patient's visit may change the behavior of the patient beyond just traditional personality measurement. Thus, while such systems may serve to improve a patient's overall experience across a number of visits, they may not serve to provide valuable insight to medical care personnel for how to interact with the patient at any specific visit. Thus, these types of systems still provide a lack of instructions to healthcare personnel for how to deal with a particular patient in a particular circumstance.

### SUMMARY OF THE INVENTION

The following is a summary of the invention, which should provide to the reader a basic understanding of some aspects of the invention. This summary is not intended to identify critical elements of the invention or in any way to delineate the scope of the invention. The sole purpose of this summary is to present in simplified text some aspects of the invention as a prelude to the more detailed description presented below.

Because of these and other problems in the art, described herein, among other things, are systems and methods that is designed to capture the context of the patient's visit along with a general categorization of their personality for the purpose of identifying actions a healthcare provider can use during the specific visit to improve the patient's overall satisfaction with their care and specifically with that visit through better understanding, better engagement, improved trust, and better overall experience with healthcare providers.

The systems and methods are particularly designed to improve reported patient satisfaction with regards to interaction with medical personnel and/or a medical procedure by capturing the context of the patient's visit using a plurality of electronic medical record, financial, and social data and the patient's personality style using input from the patient and others and to identify a selection of interaction recommendations for use during the visit that, when used by the healthcare provider, improves the patient's overall experience and satisfaction with the visit. This, in turn, allows for healthcare providers to provide more personalized and personable care which has been shown to improve patient satisfaction and can result in increased care compliance and overall care outcomes.

In order to implement the above systems and methods, the systems and methods may be embodied into a computer system as well as computer implemented instructions provided on transitory or non-transitory media for providing healthcare providers patient engagement suggestions or “actions” which are based on the correlation of the action to the context of the patient visit and the patient's personality style. It is believed that use of the actions results in an improved patient experience and satisfaction. In an embodiment, the system and method comprises a process for data collection that includes electronic integration to 3^{rd} party systems including electronic medical record (“EMR”) systems among others, and user interfaces that enable data collection; a process for data capture of a patient's personality style through electronic integration, integration with social media and other online environments, and through manual entry through a user interface; and an action identification that chooses a set of the most relevant actions through analysis of plurality of available actions and using patient personality and visit context and the relevance of an action to the characteristics of the individual personality and life situation traits specific to a visit or trended over many visits.

In some embodiments, there is provided a patient satisfaction analysis tool that uses regression analysis as a primary method to evaluate the actions chosen based on the patient information to the patient's stated level of satisfaction. Healthcare provider performance data is also provided to the health system along with similar regression analysis to characterize the patient/provider relationship and potential efficacy.

In still further embodiments, there is provided machine learning capability that analyzes current and historical patient satisfaction data along with the variables related to the patient personality and context of visit and can perform extended regression analysis to test for correlation levels based on a configurable level to identify areas for potential improvement. The system applies regression analysis, scenario analysis and others to identify material changes to the use of variables, characteristics, weights of variables, and actions and then implements these changes either in real-time or via scheduled updates. Through this, the system will continually learn and improve the accuracy of the system.

There is described herein, amongst other things, a method of providing automated suggested actions for a healthcare provider during a patient visit, the method comprising: having personality information on a patient, the personality information being stored in a database accessible to a computer; having visit information relating to a patient visit, the visit information being stored in a database accessible to the computer; the computer automatically using the personality information to develop a Patient Personality Type (PPT) for the patient; the computer automatically using the visit information to develop a Visit Lifecycle Context Rating (VLCR) for the visit; the computer automatically using the PPT and the VLCR jointly to locate a first group of actions from an action matrix; the computer automatically using specific elements of the personality information and the visit information to choose selected actions from the first group of actions; and the computer automatically providing the selected actions to a healthcare provider at the initiation of the patient visit.

In an embodiment, the method further comprises the computer obtaining feedback on the use and effectiveness of the selected actions at a time after the initiation of the patient visit.

In an embodiment, the method further comprises the computer utilizing the feedback to alter how the computer chooses or weights the specific elements of the personality information and the visit information to choose selected actions from the first group of actions

In an embodiment, the method further comprises the computer utilizing a machine learning engine.

In an embodiment, the method further comprises the selected actions includes providing the selected actions part of an electronic medical record (EMR).

### BRIEF DESCRIPTION OF THE DRAWINGS

FIG. 1 provides a general block diagram of an overview of an action generation and utilization system.

FIG. 2 provides a block diagram of an embodiment of a system providing an example of the ways a patient's personality (“Patient Personality Type” or “PPT”) may be captured and categorized along with a method by which the system may develop the context of the visit (“Visit Lifecycle Context Rating” or “VLCR”).

FIG. 3 provides a block diagram of an embodiment of a system flow that describes how an embodiment of the system uses the PPT along with the VLCR to identify specific actions it will provide to healthcare providers when working with a specific patient under certain circumstances. The diagram also indicates how these actions may be provided to the healthcare provider for their use.

FIG. 4a provides an embodiment of a VLCR scale that may be used to determine the variables of the VLCR value of an action matrix.

FIG. 4b provides an embodiment of an application of the action matrix along with examples of the relevant actions that could be chosen.

FIG. 5 provides a screenshot of an embodiment of a patient's Electronic Medical Record (“EMR”) showing action recommendations in an embodiment of the system.

FIG. 6 provides a block diagram of an embodiment of a system or process of capturing patient satisfaction and using that data to analyze the efficacy of the system and healthcare provider performance. It also shows how the analysis may be used to create a data repository for use with the machine learning module.

FIG. 7 provides a block diagram of an embodiment indicating how a machine learning module can use the outcome and satisfaction data along with other data available to validate and improve the overall system.

### DETAILED DESCRIPTION OF THE PREFERRED EMBODIMENT(S)

Developing a system that can create actions for the healthcare provider based on the intersection of the patient's specific personality and the visit context can create an improved method of interaction with the patient during a specific visit. This can be more effective to improving patient outcomes and satisfaction both with that specific visit and overall. In addition, the use of machine learning or related methods to test the efficacy of such a system in terms of the value and use of personality indicators, the myriad of ways to consider a visit in context, how to make recommendations for improved correlative actions to results, and the ability of the system itself to find ongoing improved correlations that improve the system's ability to pin-point specific care actions, provides for a potential to dramatically improve interactions within healthcare settings and to improve patient compliance with proposed care.

The systems and methods preferably use a machine teaming engine or similar system, process, or method to further evaluate the system for improvements in use, weighting, and context of variables, relationship structures and other elements that will lead to the system improving its action prediction capability.

It should be recognized that the disclosure herein is focused on patient satisfaction in the medical field and in conjunction with medical procedures, interactions, and treatment. This is by no means required and while this is a valuable exemplary embodiment, one of ordinary skill in the art would understand that such field of use is by no means required and the systems and methods may be used in other contexts and fields where satisfaction of a customer or other individual is desired. This can include, but is not limited to, government, public utilities, law, private businesses, and educational settings.

Further, this description and disclosure illustrates by way of example and not by way of limitation. This description will clearly enable one skilled in the art to make and use the disclosed systems and methods, and describes several embodiments, adaptations, variations, alternatives and uses of the disclosed systems and methods. As various changes could be made in the above constructions without departing from the scope of the disclosures, it is intended that all matter contained in the description or shown in the accompanying drawings shall be interpreted as illustrative and not in a limiting sense.

Throughout this disclosure, the term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, smart phones, tablet computers, mobile devices, server farms, hardware appliances, minicomputers, mainframe computers, video game consoles, handheld video game products, and wearable computing devices including but not limited to eyewear, wristwear, pendants, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the term “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service, or other distributed or collaborative computer networks.

Those of ordinary skill in the art also appreciate that some devices which are not conventionally thought of as “computers” nevertheless exhibit the characteristics of a “computer” in certain contexts. Where such a device is performing the functions of a “computer” as described herein, the term “computer” includes such devices to that extent. Devices of this type include but are not limited to: network hardware, print servers, file servers, NAS and SAN, load balancers, and any other hardware capable of interacting with the systems and methods described herein in the matter of a conventional “computer.”

A specific type of computer is a “mobile device” which is a computer which is generally designed to be transported with and associated with an individual user. A “mobile device”, thus, typically includes association with a user as well as association with a particular location in physical space, such as through the Global Positioning System (GPS). The term “mobile device” as used herein includes, but is not limited to, devices commonly referred to as smartphones, tablet computers, and wearable computers.

Throughout this disclosure, the term “software” refers to code objects, program logic, command structures, data structures and definitions, source code, executable and/or binary files, machine code, object code, compiled libraries, implementations, algorithms, libraries, or any instruction or set of instructions capable of being executed by a computer processor, or capable of being converted into a form capable of being executed by a computer processor, including without limitation virtual processors, or by the use of run-time environments, virtual machines, and/or interpreters. Those of ordinary skill in the art recognize that software can be wired or embedded into hardware, including without limitation onto a microchip, and still be considered “software” within the meaning of this disclosure. For purposes of this disclosure, software includes without limitation: instructions stored or storable in RAM, ROM, flash memory BIOS, CMOS, mother and daughter board circuitry, hardware controllers, USB controllers or hosts, peripheral devices and controllers, video cards, audio controllers, network cards, Bluetooth® and other wireless communication devices, virtual memory, storage devices and associated controllers, firmware, and device drivers. The systems and methods described herein are contemplated to use computers and computer software typically stored in a computer- or machine-readable storage medium or memory.

Throughout this disclosure, terms used herein to describe or reference media holding software, including without limitation terms such as “media,” “storage media,” and “memory,” may include or exclude transitory media such as signals and carrier waves.

Throughout this disclosure, the term “network” generally refers to a voice, data, or other telecommunications network over which computers communicate with each other. The term “server” generally refers to a computer providing a service over a network, and a “client” generally refers to a computer accessing or using a service provided by a server over a network. Those having ordinary skill in the art will appreciate that the terms “server” and “client” may refer to hardware, software, and/or a combination of hardware and software, depending on context. Those having ordinary skill in the art will further appreciate that the terms “server” and “client” may refer to endpoints of a network communication or network connection, including but not necessarily limited to a network socket connection. Those having ordinary skill in the art will further appreciate that a “server” may comprise a plurality of software and/or hardware servers delivering a service or set of services. Those having ordinary skill in the art will further appreciate that the term “host” may, in noun form, refer to an endpoint of a network communication or network (e.g., “a remote host”), or may, in verb form, refer to a server providing a service over a network (“hosts a website”), or an access point for a service over a network.

Throughout this disclosure, the term “real time” refers to software operating within operational deadlines for a given event to commence or complete, or for a given module, software, or system to respond, and generally invokes that the response or performance time is, in ordinary user perception and considered the technological context, effectively generally cotemporaneous with a reference event. Those of ordinary skill in the art understand that “real time” does not literally mean the system processes input and/or responds instantaneously, but rather that the system processes and/or responds rapidly enough that the processing or response time is within the general human perception of the passage of real time in the operational context of the program. Those of ordinary skill in the art understand that, where the operational context is a graphical user interface, “real time” normally implies a response time of no more than one second of actual time, with milliseconds or microseconds being preferable. However, those of ordinary skill in the art also understand that, under other operational contexts, a system operating in “real time” may exhibit delays longer than one second, particularly where network operations are involved.

In an embodiment, the systems and methods contemplated by this disclosure will be embodied in a computer system running appropriate software to analyze a patient's context of their visit and current life along with their underlying personality to provide a set of specific engagement actions to a healthcare provider that are indicated to improve the patient experience during their care, visit, and their resulting patient satisfaction. The computer systems typically capture all necessary data both automatically from available resources as well as from manual data entry during a patient visit and identifies and automatically communicates the specific actions for a healthcare provider to take during a specific visit based on the nature of that visit and the specifics of the patient.

Embodiments of the system also measure patient satisfaction results from accepted satisfaction surveys in a way that provides performance data for healthcare providers which is useful and actionable. Data across the entire system may be applied to a machine learning engine or similar system that identifies effective and ineffective data relationships at a macro level and then determines what changes may be made to improve the relationship both with a specific patient and/or with patients that are deemed similar to the specific patient at a more macro level. The systems and methods can also identify effective and ineffective actions and potential changes to improve performance.

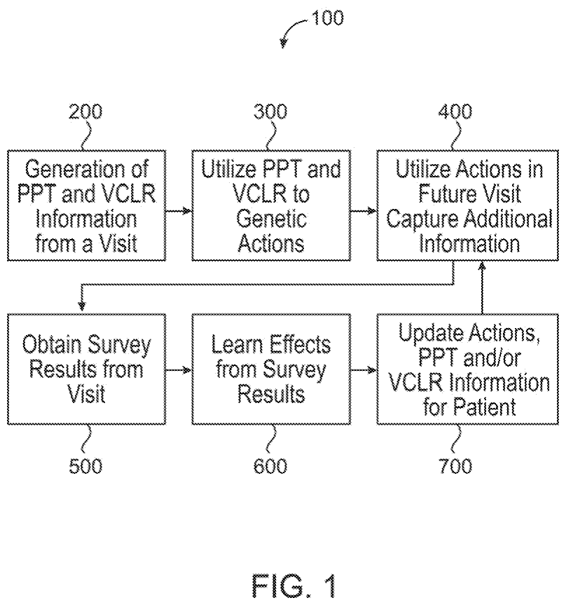

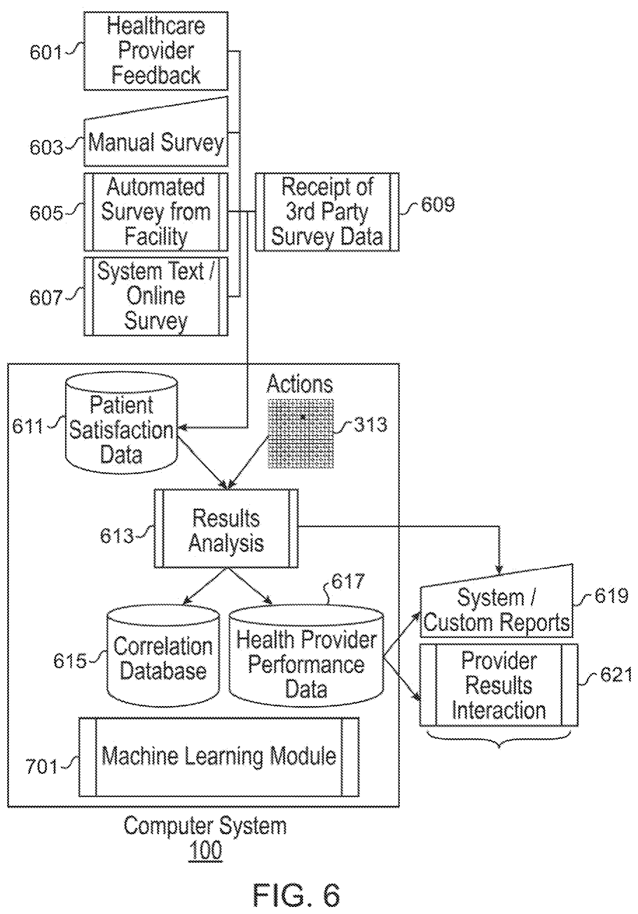

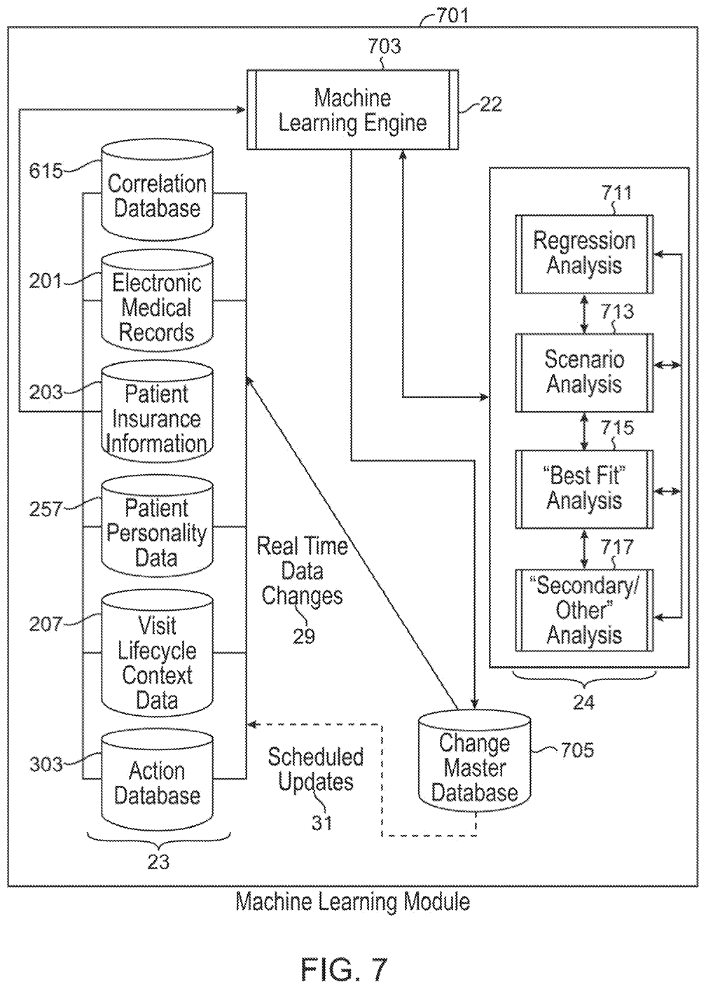

FIGS. 1-7 provide for general block diagrams depicting an embodiment of a system (100). The system (100) can serve to provide specific recommendations or “actions” to take as part of a patient's care and which healthcare personnel can act on in the course of an office visit (FIGS. 1-5). The system (100 can also provide for the collection of survey information and machine learning based on that survey information (FIGS. 6-7) to test and improve the selection of actions. The system (100) will typically be built to include hardware and/or software to integrate and utilize information and data which may be provided from multiple different sources. Thus, the system (100) may include an automated integration layer to facilitate the transfer of information between internal components and external systems including systems by the same manufacturer as well as commercial 3^{rd} party and proprietary systems where it may obtain information in open or proprietary formats. This integration layer may be used to build and implement data connections between the system (100) and other systems in any manner known to one of ordinary skill in the art. These integration points may enable real-time communication or file-based integration depending on the needs of the system (100).

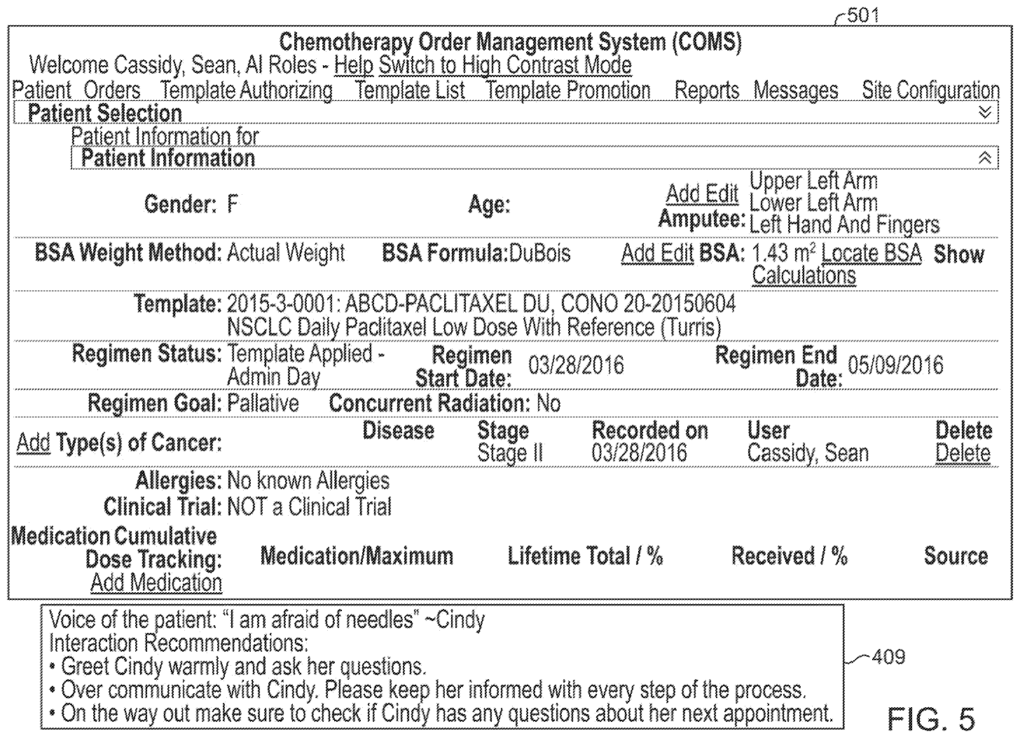

The system (100) will typically be built with human user interfaces to enable the system (100) to be configured, to be used operationally, and to be maintained. Information from the system will typically be provided as part of a display to a human user (such as is shown in FIG. 5) but the proposed actions provided by the system may be provided utilizing any form of human user interface to allow a human to act on them.

Preferably, the system (100) would be built with an integrated security layer or other security protocol or system to enable the system to comply with all regulatory security requirements in addition to normal access and control security. In the medical field, the system will generally be built with consideration and in compliance with the Health Insurance Portability and Accountability Act of 1996 (HIPAA). As such, the system (100) will generally have patient data privacy and security provisions for safeguarding all medical information. This can include, but is not limited to, encryption of stored and communicated information and password or similar protections for accessing the information or actions. The system (100) may be built using any then current distribution models that may include Software as a Service (SaaS) or “on-site” based models and/or other models that are known now or later discovered.

The systems and methods discussed herein typically relate to the functions and operation of a software system running on a computer that assesses patient's personality and uses medical records and other information to provide an understanding of the context of the patient visit and personality data to determine the personality style. Such a system will evaluate the patient personality and the context of the visit to create a specific set of actions for the healthcare provider to use when interacting with patients during a patient visit. The system can assist in correcting a concern in the operation of existing patient management or electronic medical records. Specifically, records systems, while they can provide for storage of prior information about a patient which is useful for a healthcare worker interacting with a patient, do not serve to provide for forward looking actions to improve patient satisfaction.

Patient satisfaction, as typically measured by survey information, is tied to reimbursement for medical procedures and to the likelihood of future patient interaction and patient compliance with proposed medical care. As such, improved patient satisfaction with care, like improved efficacy of a drug, will provide for concrete improvements to the effectiveness of a patent's medical care. Patients which see the care as more valuable and understand the reasons for medical decisions being made are more likely to agree with them, comply with medical care procedures, and accept necessary tradeoffs and side effects from a course of treatment.

FIG. 1 provides a general overview of the operation of an embodiment of a system (100) for providing specific recommendations (called “actions” herein) for interacting with a patient and for improving the types and nature of the supplied actions based on their efficacy for a specific patient. In the embodiment of FIG. 1, the system (100) is launched with available initial information previously collected for a patient (200) as well as information which is collected for a target visit which may be collected in real time both before and during the visit. As the system (100) is designed to utilize information specific to a patient, this information will typically be necessary to set initial values related to the Patient's Personality Type (“PPT”) and their Visit Lifecycle Context Rating (“VLCR”) for the patient for this visit. This initial generation will typically be either in conjunction with a patient's first visit to the healthcare entity that is utilizing the system (100) or first visit since the system (100) has been implemented. The generation may be done only once that visit is complete, but will typically be performed in real time or near real time from initiation of the visit and continuing while that visit is progressing.

Once the original determination for PPT and VLCR is made (200), that information may be updated as the visit progresses and will be used to determine specific actions for that patient for use in both the current and future visits (300). When a visit occurs, the actions are provided to healthcare personnel for utilization during the visit (400). After the visit (400), the patient's satisfaction level with the visit will typically be captured using survey results (500). The survey results can then be fed back into a machine learning algorithm or other mechanism to evaluate the effectiveness of the previously proposed actions (600). This can also be combined with new information which was collected during that visit. Based on what is learned from the actions' implementation (600), settings about the patient may be updated, and/or additional or alternative actions may be entered (700) in preparation for the patient's return and future visits. While FIG. 1 gives a basic overview of the process, the remaining FIGS provide for additional detail about how these elements are carried out.

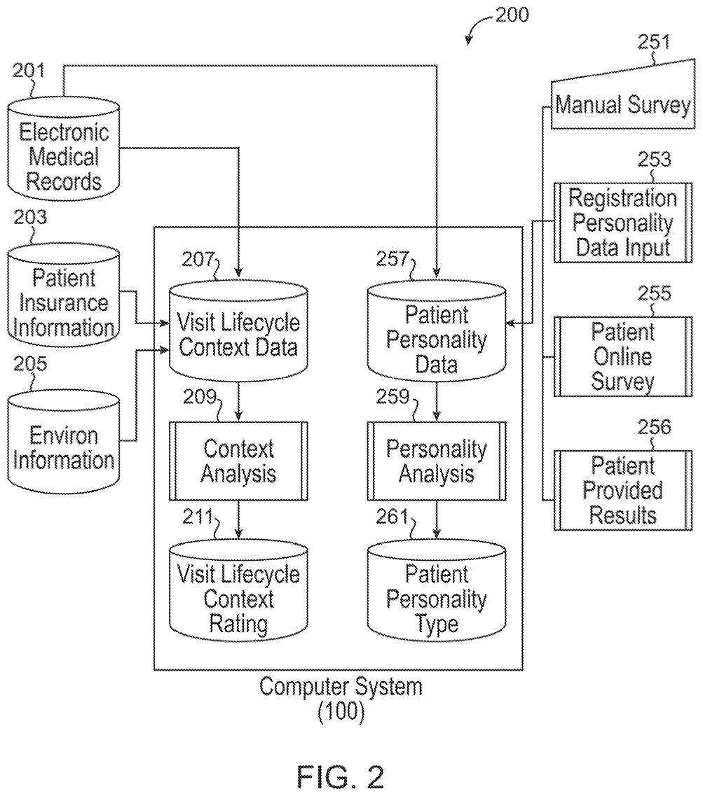

FIG. 2 discusses the generation of PPT (261) and VLCR (211) values for a specific patient (200). Generation of these values will typically be triggered by the initiation of a patient visit to the healthcare provider, however, existing background information on patients that have previously visited the healthcare entity can also be processed to generate a baseline in preparation for initiation of a new visit. The two values of PPT (261) and VLCR (211) work synergistically to assist in recognizing both a patient's general “type” for interaction with healthcare personnel as well as to weight or move actions for that type based on specific occurrences in that patient's life. This weighting is typically based around stress factors or similar elements which will alter a patient's typical personality traits based on the current environment they are in and the nature of the visit. To understand the potential stress factors that could affect the patient's personality during the visit, the system utilizes available data points to determine the context of the visit both in advance of the visit and while the visit progresses.

The context of the patient's visit is referred to herein as the patient Visit Context Lifecycle Rating (“VLCR”) (211) and is designed to take into account indicators of the level of stress (“stressors”) likely to be experienced during a specific visit based on current factors in the patient's life. This includes factors specific to the medical reason for the visit, factors unrelated to the medical reason for the visit but specific to the visit, and other general factors which may be effecting the patient at the visit which are disconnected from the healthcare entity.

To give a general idea of what a VLCR (211) is looking to capture is best done by specific example. If one was to take a generally very laid back patient when it came to medical care but require them to go to a medical visit that they felt was a waste of time, that patient will likely be less receptive to medical advice given during that visit. This comports with well understood reactions and effects of stress on learning. Further, if getting to that visit included a stressful driving situation making them late to arrive for the visit, which, in turn, resulted in delays to have the visit begin which then was making them late for an important event they had scheduled for after the medical visit, this normally very laid-back patient may be extremely hostile, adversarial, and opposed to listening to any advice during the visit.

To obtain stressors associated with the medical context of the visit, the system typically utilizes electronic medical records (201) to obtain specific information related to the patient's medical history and specific reasons for the current medical visit. Information related to medical payment may also be obtained from patient insurance information (203) as ability to pay and understanding of expected reimbursement can also act as stressors. Information may also be obtained from databases of general environment information (205). This can include things such as weather information (such as, but not limited to, difficult or dangerous local weather which may be experienced getting to the visit), news information (such as, but not limited to, protests, local contagious disease rates, political strife, or increased crime), or general population information (such as, but not limited to, local macro happiness indicators) as these can also influence stress currently experienced by a patient.

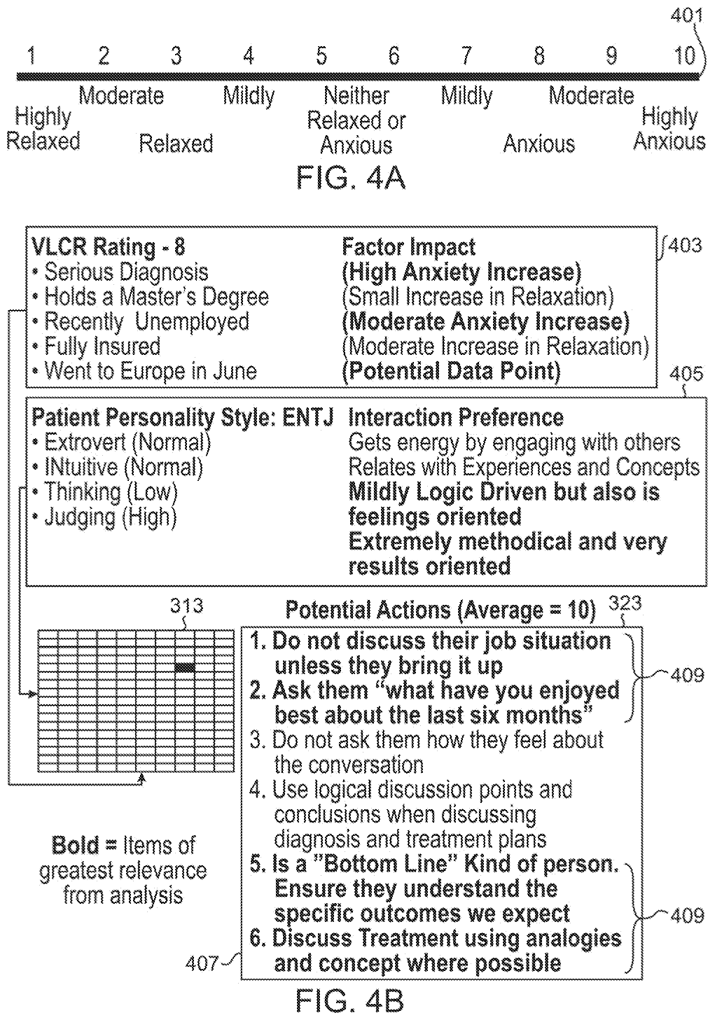

The information from these sources will typically be collated to provide data on the specific context of the selected visit (207). The system may provide context analysis (209) by analyzing the data that is collected and applying a sum of weighted variables formula based on the total number of variables collected and the associated weight of those variables to arrive at the patient VLCR (211). The system has the ability apply this formula to a dynamic, rather than static, set of variables. Selection of the VLCR (211) is discussed in greater detail in conjunction with FIGS. 4a and 4b.

A “visit” in the context of this disclosure will typically be measured from the time the patient has an initial contact with healthcare personnel that initiates the visit through the point of the patient leaving the facility, and will typically include discharge. It may, therefore, encompass a time much longer than just while the patient is in the healthcare facility as it could involve getting to the facility as well as scheduling related stressors. In this way, stressors associated with a visit which may not be specifically medical in nature, but are associated with this medical facility or this visit may be taken into account. For example, a patient is likely to be more stressed during a visit if they got lost trying to locate the facility or if they had difficulty making the appointment for the visit even though these occurred before the patient arrived. Similarly, a patient who has waited for a long time in a waiting room or experienced a problem with a receptionist may also be quite stressed from the admission experience. For these reasons, the visit lifecycle may include any in-patient or admission time in addition to the scheduled visit and whether the in-patient time was expected or not.

For these types of reasons, whenever useful information is obtained about the visit and is input into the system (100), the system (100) will typically update the actions associated with this patient in real time or near real time. Thus, should a patient who is unlikely to be stressed, for example, be likely stressed by actions or activities which take place while they are waiting for the visit to commence, they may be provided actions more appropriate for a patient with higher stress state (or more naturally stressed personality) during the visit than they would normally.

The system will typically initially be configured to integrate data related to the a) reason for the patient visit, b) patient history information related to past visits and severity of visits, and c) patient demographic information including but not limited to age, gender, religious preference, level of education, income level, current insurance coverage and limits, and employment status. However, in an embodiment of the system (100) Information utilized may include, but not be limited to: disease diagnosis, insurance, religion, payer method, sex, age, current weather, time of appointment, on-time start information, wayfinding feedback, registration process feedback and the like. Other data points may be utilized if they are found to affect either this patient's visit or patients' visits generally either positively or negatively.

In addition to generating a VLCR (211) for the patient, the system will also typically determine a PPT (261) for the patient. The PPT (261) is determined to better predict the patient's desired approach to how the patient would typically like to be engaged during the visit as well as how the patient is likely to react to the stressors captured in the VLCR (211) of the visit. As PPT (261) is specific to the patient and will often change much more slowly across visits than the VLCR (211) (which is specific to each visit) the PPT (261) will often be determined ahead of the specific visit based on prior information about the patient. However, the PPT (261) may also be obtained during a visit (e.g. while waiting) particularly if no PPT (261) had been previously established. The PPT (261) will typically be generated from a variety of information provided such as, but not limited to, through patient surveys while waiting (251), patient provided information during registration procedures or from reactions noted by medical personnel during registration procedures (253). Patient surveys performed in advance of the visit (255) may also be used as may a patient providing results of personality assessment from elsewhere (256). These tests will often provide indicators as to the strength of one given trait in a way that would affect behavior when compared to others in certain circumstances.

Specific patient personality test data can be gathered by the system through personality data inputs which may be gathered in a number of different ways including, but not limited to, a) a patient's response to a standardized personality test that they complete in full based on the health system's external patient marketing and wellness programs or similar; b) a patient's response to a standardized personality test provided through social media that is able to be captured by the healthcare system as part of its social media presence or via data mining techniques; c) a patient's response to electronic media including text messages where the patient is provided a text of different kinds of personality questions over time with the reply to the message being captured and used to develop a personality profile over time; d) a questionnaire completed during patient intake or during a wait room visit that provides enough insight to make a personality type determination; and/or e) as a default personality type based on information gleaned from the health record to include the use of the context of the patient's visit to make a generalized personality assessment.

The system (100) once it has the various forms of data will then collate the data (257) for analysis (259). Analysis of personality data (259) will typically be against the personality method and rules being used and derive the PPT (261) based on a “best fit” formula that matches patient personality data to personality traits pre-configured in the system to derive a PPT (261). For patients that provide a known personality type, such as by completing a specific known personality test (251), the system will have the ability to store that outcome directly as the PPT (261).

For purposes of determining and capturing the PPT (261), an embodiment of the system may utilize accepted personality indicators from accepted personality tests such as, but not limited to, the DiSC personality indicator, 16PF, Hexaco Model, Revised NEO Personality Inventory (NEO-PI-R), Myers-Briggs personality indicator, Eysenck Personality, Minnesota Multiphase Personality Inventory (MMPI, MMPI-2, or MMPI-2-RF), The Birkman Method, or any other test, methodology, or criteria known now or later discovered. As indicated, if data is captured (257) which is specific to one of these tests, little analysis (259) may be required and the result may simply to assigned to the PPT (261). Alternatively, unspecific data may be analyzed (259) to capture a representative personality indicator indicative of multiple traits and a composite overview of the patient along with relevance and influence of any individual components on the patient's particular style, engagement, understanding, and/or response. From this, analysis (259) may infer a specific PPT (261) based on a preferred indication test. Similarly, should formal personality information be provided by a patient from one type of personality test (257), that data may be analyzed (259) to generate a personality type in a preferred form based on comparisons between tests in the generation of the PPT (261).

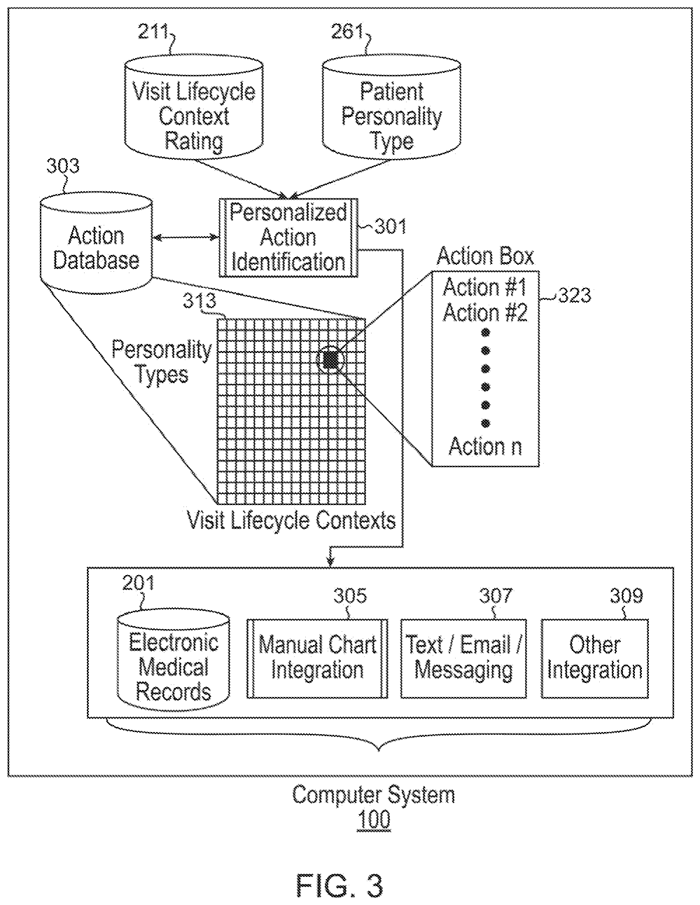

With determination of the PPT (261) and VLCR (211) for this particular visit having been calculated, the system (100) will then move on to FIG. 3 to compare these variables and identify an intersection point enabling the system (100) to identify and array (323) of potential actions to be taken during the visit. Certain selected actions can then to provided to the healthcare provider based on the makeup of the patient prior to the visit allowing the healthcare worker to act on them during the visit.

In FIG. 3, the two variables of VLCR (211) and PPT (261) captured in FIG. 2 are matched together based on their intersection in a matrix (313) which provides connection to a database of configured actions (303). Each interception point in the matrix (313) forms an “action box” (323) where each action box (323) contains default actions (407) that may be selected (409) and then used by the healthcare provider to govern the style and approaches used during the visit as shown in FIG. 4b. The selected actions (409) will be provided to medical personnel in a variety of methods or formats and can include providing them as annotations to a manual medical record (305) or an Electronic Medical Record (EMR) (501) in database (201) which is shown in increased detail in FIG. 5. Alternatively, the selected actions (409) may be provided to the personnel more directly such as via an SMS text message, email, or similar communication (307) directly to healthcare personnel's devices. This direct communication (307) may be preferred if real time data indicates a major change for a patient immediately prior to a visit or during a visit where the medical personnel may not receive the action change otherwise (e.g. if an EMR (501) is not being accessed at the time). The system also contemplates other forms of action provision (309) using other known or later developed technologies appropriate for such communications. Medical personnel then use these selected actions (409) to enable better dialogue, understanding, patient comfort and trust, and other factors that will affect the patient experience and ultimate satisfaction.

FIG. 3 provides an embodiment of the basic process by which the system (100) arrives at an appropriate set of selected actions (409) for use by the healthcare provider. The system (100) will typically have an action database (303) that is the core repository of all possible actions that could be provided to a given healthcare provider. These are typically developed in advance and are designed to take into account a potential world of actions. In most cases, the actions will exist as both an affirmative and a negative of the same action. For example, an affirmative action for a patient may be to provide test data to a patient in raw form while the corresponding negative action would be to not provide test data in raw form or to provide data in a processed form. There is also an inferred third version of the same action which is the neutral of the affirmative and negative of the action. This would typically be represented by no presentation of an action related to his particular element the neutral indicating that there is no preference as to action. In the above example, saying nothing about data presentation would be a neutral action. In a neutral action situation, the healthcare provider is essentially allowed to utilize their own judgement as to if (or how) to act (in this case in what form to provide data).

The core of the action database (303) involves the action database matrix (313) which provides a data point for an intersection of the specific VLCR (211) and the PPT (261) for the patient. While the matrix (313) of FIG. 3 is shown as two dimensional, this is by no means required and the intersection of the VLCR (211) and PPT (261) may actually be a multi-dimensional matrix with any number of dimensions. The configuration of the matrix (313) will be based on the range of possibilities available and required in the VLCR (211) and the PPT (261) and each intersection will typically not provide a single action, but will provide an array (323) of actions (407) for that specific patient. These will typically be a mix of positive and negative actions. An action (407) not being in the array (323) effectively provides a neutral action on that element.

FIGS. 4a and 4b provide for an embodiment of operation of the matrix (313) in more detail. In FIG. 4a, the VLCR (211) is provided as a scalar value (401). In this embodiment, the scalar value is a ranking from 1 to 10. The range in this embodiment is based on a 10 point integer scale and the deviation from a patient condition “5”, or expected norm, being neither relaxed or having particular anxiety about the upcoming visit. A highly relaxed attitude will typically be at the low end of the scale being measured as a “1” and a highly anxious state being measured as a “10”.

While being able to select a particular single scalar value of the VLCR (211) can be useful for analysis, FIG. 4b shows how the system can identify factors (403) that are likely more increasingly driving the VLCR rating (211). The factors are typically classified as either increasing or decreasing anxiety, and are provided with a strength indication of their increase or decrease. As can be seen in the factors (403), the factors are indicated to either increase or decrease stress (anxiety) and may have a small, moderate, or high alteration. Further, certain factors may not create or decrease anxiety, or are unknown in effect, but may be useful as data points for getting more information.

In the factors (403), an indication of the serious nature of a prior diagnosis which is likely to be the major purpose of the visit (for example, a potentially life threatening disease or an injury requiring major intervention) is categorized as a high increaser of stress. Similarly, the patient's recent unemployment event is also likely to increase stress, but probably not as much as the diagnosis. Further, these two items in conjunction may be seen as strongly anxiety inducing as the patient may have high concern about paying for expensive, but recommended, procedures or medications, or from having significant downtime which could postpone a job search.

The factors (403) also provide for data which indicates a decrease in anxiety. For example, as the patient holds a master degree, they may be more likely to understand the necessity and benefits for complicated treatments and may be better able to understand statistical outcomes and risk evaluation. They may also have a better understanding of the serious diagnosis from personal research where understanding can decrease anxiety. Further, the indication that the patient is fully insured (even with the recent unemployment) can provide for a reduction in anxiety over paying for procedures. In addition to these specific stressors, the factors (403) have also identified a data point the effect of which may not be readily determined, or which may be of value as a conversation piece. In particular, the factors (403) indicate that the patient recently took a vacation to Europe. This, and other data points such as obtaining a new pet, being mentioned in the news, or publishing a book, among others, are likely to be positive with regards to anxiety, but not directly. In particular, such factors (403) may provide a method to reduce expected anxiety as well as connect with the patient at a more personal level. However, they could also be stressors.

Combined with the VLCR (211) in the matrix (313) is the PPT (261). The PPT (261) may also be displayed as a scalar value using an arrangement similar to that of FIG. 4a but ranking between, for example, a generally relaxed to a generally anxious personality. Often the PPT (261) will not be a single scalar value, but a category. For example, the PPT (261) may be based on the ordinal range or specified category measurement of the personality mythology that is being used for the system. In the PPT (261) of FIG. 4b, the Myers-Briggs method is used for analyzing the PPT (261). Myers-Briggs method utilizes 4 core personality traits (each of which has two possible values) with the combination presenting one of sixteen categories that are the sum of the combinations of each pair of the four core personality traits. In the Myers-Briggs method, the patient's core personality traits are based on them being generally: Introvert vs Extrovert, Intuition vs. Sensing, Thinking vs Feeling, and Perceiving vs Judging. Each of the traits then generally serves to give the person a dominant four letter score based on which direction they prefer in each category. For example, a patient preferring the first of each of the four categories would be considered INTP. The score is then typically weighted as to how much toward each of the directions the patient leans.

In the embodiment of FIG. 4b, the patient is indicated to be ENTJ as shown. However, the weighting shows that while the patient is classified as extroverted and intuitive, those are not strongly or weakly presented and likely won't strongly influence their reaction to the stressors identified by the VLCR (211). However, their thinking classification is low and their judging is high. These latter two are more likely to influence their personality uniquely and so the specific influence of them is likely to have more impact in the matrix (313) analysis.

With the PPT (261) and VLCR (211) provided, these are placed in the action database matrix (313) to identify the intersection point of the variables within the matrix (313). In this case, the action box (323) selected is based on a PPT (261) of ENTJ and a VLCR of 8. This selects a collection of possible actions (407). It should be noted that the array of actions (407) shown in FIG. 4b is not necessarily complete, but is a representative sample. All of these actions may be presented to medical personnel. However, in an embodiment, the system (100) evaluates the possible actions (407) and only selects the most relevant. This selection of relevancy is indicated by the use of bold text in the VLCR factors (403), PPT calculations (405), and possible actions (407).

In particular, in this embodiment, the system has determined the PPT (261) includes personality factors which are stronger in one area than another as discussed above. For instance, in this example while the patient is classified utilizing a specific overall personality type, the system (100) has also noted that the patient is more balanced in certain areas and is typically classified within a normal range or most categories. However, the patient does have certain traits which are more emphasized or expressed more strongly. Thus, while the possible actions (407) are generally laid out by intersection in the matrix (313), the system uses more relevant elements of the VLCR (211) and PPT (261) to choose the most relevant actions to deliver.

The actions (407) are typically evaluated and only selected actions (409) provided to the healthcare worker based on selecting actions that are specifically relevant to the patient's makeup via the intersection of both the PPT (261) and the VLCR (211) and the specifics that went into the determination of each value. Actions may be eliminated as non-relevant or emphasized based on the high likelihood of correlation of the patient and the action to a positive satisfaction outcome. Actions will typically be used as a baseline of understanding related to patient engagement approach. The healthcare provider may also provide feedback on the relevance of indicated actions provided along with any other observations that may affect the patient's satisfaction.

One can see this is effect in FIG. 4b. In this FIG. 4b, the high stress level of their recent unemployment would mean that the topic of their job should not be discussed to avoid inducing additional anxiety during the visit. Thus, the action (407) of not discussing the job situation is selected as an action (409) to be provided. Further, while their personality indicates that they should like methods and logic, the relative position of these points more clearly indicates that they want to get to the outcome and result quickly. Thus, the action for outcomes is selected (409) while the use of logic in discussing the outcomes is not as this may take too long (making this effectively a neutral action). Finally, the European vacation as a data point provides for a potential talking point which may be useful for anxiety reduction. Thus, the selection of the action asking about recent areas of enjoyment is selected (409).

It should be noted that many of the actions (407) are not specific to the factors (403) or calculation (405) which resulted in their generation or selection. This is because the system (100) will typically not be able to recognize certain details of a factor (403) or action (407). For example, the system (100) will not necessarily be able to recognize that the patient took a trip to Europe, or even that they took a trip. However, that data point can be identified as relevant and the timing can be simply selected as recent. For example, it may have been entered as an event when the patient checked in implying it was relatively recent, and the 6 months can be selected simply as a likely relevant time period for new events of interest, or because that it's the amount of time that has passed since the patient was last seen. In any case, the action (407) related to the trip is not specific (e.g. it does not suggest talking about their European trip, or even any trips) it instead simply asks for positive events in the last 6 months. Further, as the positivity of the trip is not known (it could have been a terrible high stress activity), the data point instead provides that the action is to ask for something enjoyable in the last 6 months. While the system (100) would be cuing to have the patient talk about the European trip, if that was a terrible event, the patient will presumably simply pick a different positive event (of which the system is unaware) instead of talking about the negative trip in response to this action.

This use of more general actions provides for a useful ability to provide actions which are selected (409) because of particular events, while at the same time not necessarily providing too much detail in the selected action (409) which could result in the action being inappropriate, disconnected from the patient, or stress inducing instead of relaxing. This can itself provide new data points for selection of actions (409). While more general types of actions are often preferred, the actions can be specific particularly where the information is well known. For example, if the “serious diagnosis” of (403) was known to require a larger number of follow up visits than may be expected or that increased pain management was known to be an issue following expected surgery, that may be indicated to be brought up specifically in an action instead of just providing actions related to discussing outcomes generally.

As indicated in FIG. 3, once the actions have been selected (409), they are provided to the healthcare provider through several different forms including annotations in the patient's EMR (211) that is used by the provider(s) as part of the normal visit protocol. FIG. 5 shows one such action selection (409) where the selected actions (409) are included after notes about the specific patient in an EMR (501). In this way a healthcare provider, upon accessing the EMR (501), can immediately review the actions and utilize them throughout the course of the visit. As can be seen from the display of selected actions (409) in FIG. 5, these selected actions (409) can be used at all stages. Thus, when this patient “Cindy” checks in, the receptionist can be well aware that “Cindy” should be strongly engaged and wait times should be communicated along with indications of what is happening at regular intervals. When “Cindy” sees a nurse, the nurse will likely want to inform her of what is going to take place in the visit early in the visit. A doctor seeing her may also provide substantial detail of what will occur during the visit or will be needed in separate visits and make sure that questions are answered before the doctor leaves her presence.

Integration of the system (100) into the hospital's EMR system is highly beneficial as it not only allows for provision of the selected actions (409), but provides valuable information in determining the context of the visit as contemplated previously. The patient's EMR data points can also be accessed by the system and will be available for future evaluation and regression for actions and for other use by the machine learning engine (703) as discussed later.

While the EMR (501) can be a particularly useful system for providing the actions, it is not the only one and alternative systems may be used. These can be particularly useful where healthcare personnel utilize different forms of record keeping or where communication via an EMR (501) may not be sufficiently swift or pervasive to be used in this case. These can include, but are not limited to, the delivery of electronic messages containing the actions to smart devices that may be used with patient interactions, printed instructions to be annotated and added to a paper based chart system, or other integration points into systems used by healthcare providers.

During and/or after the visit, an embodiment of the system (100) will utilize the specifics of this visit to understand the patient's perception of the visit. Further, the system (100) may engage the patient for patient satisfaction evaluation before and during their encounter as well. This evaluation of the results is discussed in conjunction with FIGS. 6 and 7.

During the visit, the healthcare provider will typically use the provided actions (409) as part of their interaction and will provide feedback (601) on the provided actions (409) based on their interaction with the patient by using a method to signal whether the action (409) was used, was relevant, helped or hindered with patient engagement, or was otherwise warranting of feedback. If an electronic method is used, the same integration used to provide the action (409) will typically also be used to gather the feedback. For example, the healthcare provider using the actions as part of an EMR (501) may provide indications of relevance of use directly to the EMR (501) via a feedback collection mechanism such as a follow up survey or via provided feedback mechanisms as part of the EMR (501). If the selected actions (409) were provided for manual use, the healthcare facility will typically provide a process to gather the feedback (601) and place it in a form useable by the system (409).

While feedback (601) on the usefulness of the actions by healthcare providers is valuable as it can show both that the providers are utilizing the actions (409) and how they think the actions (409) are interacting with the patient at the time, the goal of the actions (409) is ultimately to improve the patient experience and satisfaction. For this reason, the system (100) will also typically quantify the patient's satisfaction of the visit by providing a specified survey instrument (603), (605), or (607) to the patient or through the integration of patient satisfaction data from a 3^{rd} party system (609). The survey instrument may be of any form including manual surveys (603), automated surveys (605) such as, but not limited to, via kiosks, or online or text surveys (607) which are sent to a patient controlled device.

Customer satisfaction survey data may be collected, depending on embodiment, from system based surveys, general health system surveys, or 3^{rd} party surveys. Data will typically be integrated into the system in a common fashion for any third party surveys used. The system (100) provides regression analysis of the patient satisfaction to the variables that lead to the actions used in the system (100) to evaluate for high or low correlation of variables. Results are stored in a correlation database (615). The system (100) also provides healthcare provider performance information through reports and interactive data use. This data is used to understand the areas of needed improvement and incentivize healthcare providers to be engaged in the patient's visit.

If the system (100) generates the patient survey it may be used at any time including before, during, or after the completion of the visit. The survey will typically have questions specific to the patient's satisfaction based on a universal set of basic satisfaction questions along with questions drawn from the relevant actions (409) provided to the healthcare provider. The surveys may be provided in several ways including paper based, electronic, text based, using social media, or other similar methods. Patient responses will typically be digitized upon receipt and will be recorded as patient satisfaction data (611).

Patient satisfaction may include factors such as the patient's comfort with the process and the interactions with medical personnel, the patient's understanding of the procedures, diagnosis, and implications of their health condition, the patient's level of compliance with the treatment approaches and protocols, the patient's current anxiety about the ability to pay for care, and their overall impression of the health system as an advocate for them and their health. The improvement in patient satisfaction may be measured through standardized methods of satisfaction used by the healthcare community including such methods as the Centers for Medicare and Medicaid Services (CMS) survey tools, private healthcare survey tools, healthcare organization designed survey tools, and/or survey tools developed and deployed specifically by this system (100).

The patient satisfaction data (611) is most useful when it is correlated with the selected actions (409) and the VLCR (211) and PPT (2671) information and the action matrix (313) that went into selecting the selected actions (409). Thus, the patient satisfaction data is typically interpreted in the context and personality style of the patient to correlate the efficacy of the derived context, personality style, and actions used to create the patient experience during this visit. This includes factors like the patient's level of satisfaction as determined by a satisfaction survey, the compliance level of the use of the actions during the visit lifecycle, and any perception or experience data the patient communications during the visit lifecycle that is captured and recorded by any caregiver during the visit lifecycle. Resultant patient compliance with proposed courses of treatment may also be evaluated as part of patient satisfaction as can whether a patient was even willing to complete a satisfaction survey. It is well-known, for example, that surveys are more likely to be completed by those who had highly positive or negative feelings toward the event than those that are more in the middle.

In further examination of FIG. 6 we see how the system will evaluate the patient satisfaction data (611) to accomplish several purposes. In the first instance, the satisfaction data (611) may be analyzed (613) and provided directly as a reporting tool (619) to provide the health system with performance data on their patient's overall satisfaction with the visit and associated care. This may be an internal report on performance or may be provided in the form of any necessary external reports to provide information to, for example, third party payers for the health care services. The system (100) will be able to provide reporting that will typically include, but not be limited to, performance based on any single visit. Alternatively, the visit information may be collated with information about the health provider from prior visits, (617) to provide macro data reports (619) on the performance of the healthcare provider. Results are reported so the provider can understand the impact of the actions on satisfaction both specifically and generally and the reports (619) and the database of information (617) may be used to provide interactions (621) between healthcare providers and the system to improve care and satisfaction generally.

As should be appreciated, depending on what is available for healthcare provider information (617) generally, the system (100) can provide reporting on specific visits, multiple visits by an individual patient or sets of patients based on any PPT (261), VLCR (211), or any specified combination of the two for both reports (619) and system interaction (621). In addition, the system (100) will typically be able to report performance data based on a single member and up to a full team of providers to improve feedback accuracy. Also, data can be compiled and reported out at a hospital or health system level, if needed. Survey results may be collected cumulatively based on the patient and their number of visits, by each provider, both on an individual point of care event and a cumulative trend of performance, and by a patient-provider pair. The patient-provider pair enables the system to understand not only the patient satisfaction and the provider's level of impact to that satisfaction, but also the compatibility of the paired encounter.

Typically, in results analysis (613), the patient satisfaction data (611) will be used as part of a regression tool to evaluate the correlation between the reported satisfaction level of the patient and the actions selected (409). This can be done as an individual selected action (409), as a “group” of all selected actions (409) or even as the general group of actions (407) including those that were not selected. In the case of an individual selected action (409), the system (100) will typically evaluate (613) general correlation and specific correlation of the action (409) to patient satisfaction. A question on the customer satisfaction survey (603), (605), or (607) may be specifically selected and placed to test the correlation of the specific question's answer to a relevant action (409).

The survey (603), (605) or (607) may also include correlation of the customer satisfaction data (611) with the selection of the PPT (261), VLCR (211), or the combination of the two via the action matrix (313). Specifically, the customer satisfaction may be interrogated to measure the impact of the PPT (261) versus the impact of the VLCR (211), for example, to accentuate the “relax” factors and neutralize or mitigate the “anxiety” factors. This may be principally measured by evaluating answers to specific questions that target the impact of the factors. In addition, overall satisfaction levels will be considered a general positive outcome related to all factors used in correlation analysis. All correlation results will typically be stored in a correlation database (615) which may include results both from this health provider and for other health providers where the information can be used to improve system (100) functionality generally. The correlation database (615) may be integrated to assist in developing the action matrix (313) and the available actions (303).

To reinforce the correlation of patient satisfaction to the actions, to identify other relevant data elements (including other actions) which use will increase potential correlation, and to continue to refine the system to provide actions that lead to improved satisfaction, the system (100) may either recommend or unilaterally adjust the collection and use of variables and the application of variables, to include weighting of variables, to improve the effectiveness of the actions (409) used with patients, and allows the user to rewrite actions (407) for specific combinations of VLCR (211) and PPT (261).

In an embodiment, the system (100) may utilize a machine learning or artificial intelligence module (701) to improve performance over time. The machine learning engine (703) will typically be used to enable the system to improve the effectiveness of data elements, improve the choosing of actions (409), experimentation with scenarios such as different personality methods, additional or changed actions (409), and other data analysis or experimentation to understand the impact of the system (100).

At the core of the learning capability of the depicted embodiment is a machine learning module (701) whose operation is shown in increased detail in FIG. 7. The system (100) will be able to integrate any existing or new machine learning engines (703) into the system for its use, including 3^{rd} party, internally developed or open source engines. The machine learning engine (703) will preferably, but not necessarily, be built to use all data available in the system (100) that relates to the patient and their visit context along with all actions (407), selected actions (409) or other items in the action database (303). The machine learning engine (701) may also use the raw inputs such as the EMR database (201) and patient insurance information (203). The VLCR factors (403), PPT calculations (405) and their underlying values (211) and (261) as well as the raw visit (207) and personality data (257) may also be used. Further, patient satisfaction data (611) and correlation data (615) will also typically be used. As illustrated in FIG. 7, access to various pieces of this data may be provided by access to the various databases where data has been stored along the way. In this way, the machine learning engine (703) can evaluate all stages of the action selection and use process.

The machine learning engine (703) will typically apply different learning methods and analysis to understand the effectiveness of the system-based current, derived, or modeled levels of correlation to the various data relationships in the system (100) seeking to optimize the effectiveness of the system (100) by adjusting various variables in the data, changing weights of variables that use a weighting scheme, increasing or decreasing the emphasis of the conditions by which the system chooses actions (409), and by modeling new and changed actions (407) to evaluate their impact. The system (100) can also use the machine learning engine (701) to adjust the action matrix (313) and even the methods used in determining the VLCR (211) and PPT (261) values and input variables. While not intending to be limited to current machine learning methods, an embodiment of the system (100) will utilize the following as the primary learning methods.

Regression analysis (711) will generally be a primary method of learning by the system in the depicted embodiment for interpreting patient satisfaction as discussed earlier. In addition, the machine learning module (701) will preferably have the ability to analyze any pair of variables to evaluate their correlation to each other and to any other configuration of data. The results of this correlation may be stored in the correlation database (615) for future use. Based on the findings of the regression analysis, the machine learning module (701) will typically create a list of improvements to data elements that are expected to result in an improved system.

Scenario analysis (713) will be used by an embodiment of the machine learning module (701) to evaluate hypothesized patient satisfaction scores by testing a given set of assumptions as to the values of the data elements in the system against the history of data related to selected actions and satisfaction scores. The machine learning module (701) will generally use this method to test recommended changes to analyze and demonstrate areas of improvement. Scenarios that result in improvements will be resolved as a set of data and system changes that will be expected to lead to the improvement.

“Best fit” analysis (715) will be used by an embodiment of the machine learning module (701) to evaluate variations in responses and preferences to expand, contract, or change the number of potential choices and weights associated with those choices to identify improvements of the granularity or use of the data when the VLCR (211) and/or PPT (261) available choices do not yield a highly correlated result to selected actions (409). The machine learning module (701) will generally define a variable for the threshold of acceptable correlation or it can allow the system (100) to apply up to a six sigma (e.g. 99.9999%) level correlation as the variable value. When a lower than acceptable level of correlation is found, the machine learning module (701) may use regression analysis to analyze the related data elements that make up that variable in the specific instance evaluated and identify which collection of variables would yield an acceptable level of correlation. When found, the system may recommend additions, subtraction or changes to the existing structure of the action matrix (313).

In addition to the above, further forms of analysis (719) may be provided to incorporate other data that could be used for analysis in areas of marketing or operations, and as a tool to integrate future methods or machine learning engines that will enhance the system.

The system (100) will generally maintain a history of all previous changes made to the system (100) by the machine learning module (701) along with current recommendations for changes to the variable data to yield expected improvements. These existing and proposed changes may be maintained in a change master database (705). The system (100) will preferably maintain a current configuration of the system (100) even while changes are being proposed or suggested by the machine learning module (701) for consistency and understanding. The change master database (705) will generally maintain recommendation data that will include but not be limited to the recommendation data element, the recommended change, the status of that change (hold, approved, discarded) and the date time stamp of its insertion into the system. Current data will be a system configuration setting that has a minimum default of 30 days and can be extended to provide for slow change to the system for consistency. Data that is no longer current will generally be archived but accessible by the system (100) for records purposes.

Changes to the system (100) based on a change in the change master database (705) will typically be done via two methods. In a first method, a real time or near real time update can be used to immediately implement system recommendations into the operational system (100). When updating, the system (100) will typically execute a series of transaction updates and will change the necessary variables while the system (100) is operating. Any routines that use these variables after they have been set will subject to the new variable values and actions may be changed in real time. Actions which are currently being accessed (e.g. due to an EMR (501) being open at the time of update) may be changed instantaneously to allow them to be immediately acted upon or my not be updated until that EMR (501) is closed.

A scheduled update will be used to update the system when a specific change cannot be made in real time due to the need for the system (100) to update its structure, table size, or other configurable relationship which could result in errors during a real time update. The system (100) can also be set to implement changes for all changes using only a scheduled method which will typically be the initial default setting to reduce errors. The scheduled update can be set manually or using basic system scheduling. Scheduled updates can also be set to run in operational model using a model that updates periodically. The minimum scheduled update can be set to any length of time. The scheduled update routine will run with major system changes being implemented, followed by structural changes to the data bases and then followed data and action adds, changes and removals.

The qualifier “generally,” and similar qualifiers as used in the present case, would be understood by one of ordinary skill in the art to accommodate recognizable attempts to conform a device to the qualified term, which may nevertheless fall short of doing so. This is because terms such as “spherical” are purely geometric constructs and no real-world component or relationship is truly “spherical” in the geometric sense. Variations from geometric and mathematical descriptions are unavoidable due to, among other things, manufacturing tolerances resulting in shape variations, defects and imperfections, non-uniform thermal expansion, and natural wear. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal geometric or other meaning of the term in view of these and other considerations.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

1. A method of providing automated suggested actions for a healthcare provider during a patient visit, the method comprising:
having personality information on a patient, said personality information being stored in a database accessible to a computer;
having visit information relating to a patient visit, said visit information being stored in a database accessible to said computer;
said computer automatically using said personality information to develop a Patient Personality Type (PPT) for the patient;
said computer automatically using said visit information to develop a Visit Lifecycle Context Rating (VLCR) for the visit;
said computer automatically using said PPT and said VLCR jointly to locate a first group of actions from an action matrix;
said computer automatically using specific elements of said personality information and said visit information to choose selected actions from said first group of actions; and
said computer automatically providing said selected actions to a healthcare provider at the initiation of said patient visit.

2. The method of claim 1 further comprising:
said computer obtaining feedback on the use and effectiveness of said selected actions at a time after said initiation of said patient visit.

3. The method of claim 2 further comprising:
said computer utilizing said feedback to alter how said computer chooses or weights said specific elements of said personality information and said visit information to choose selected actions from said first group of actions

4. The method of claim 3 wherein said computer utilizing includes utilizes a machine learning engine.

5. The method of claim 1, wherein said providing said selected actions includes providing said selected actions part of an electronic medical record (EMR).
